(19) United States
(12) Patent Application Publication
(10) Pub. No.: US 2022/0087542 A1
(43) Pub. Date: Mar. 24, 2022

(54) PHYSIOLOGICAL PARAMETER MONITORING DEVICE AND METHOD

(71) Applicant: SHENZHEN MINDRAY BIO-MEDICAL ELECTRONICS CO., LTD., Shenzhen (CN)

(72) Inventors: Zehong GUAN, Shenzhen (CN); Wenyu YE, Shenzhen (CN); Zehui SUN, Shenzhen (CN); Sanchao LIU, Shenzhen (CN); Zuming YAO, Shenzhen (CN)

(73) Assignee: SHENZHEN MINDRAY BIO-MEDICAL ELECTRONICS CO., LTD., Shenzhen (CN)

(21) Appl. No.: 17/478,838

(22) Filed: Sep. 17, 2021

(30) Foreign Application Priority Data
Sep. 18, 2020 (CN) 202010990160.0

(51) Int. Cl. A61B 5/0205; A61B 5/0215; A61B 5/1455; A61B 5/318; A61B 5/00

(52) U.S. Cl. CPC A61B 5/0205; A61B 5/0215; A61B 5/72; A61B 5/318; A61B 5/14551

(57) ABSTRACT

Disclosed are a physiological parameter monitoring device and method. The monitoring device includes a front-end circuit, a processor, and an output component, where the front-end circuit is configured to acquire a physiological signal from a signal output by at least one sensor for sensing a physiological signal of an organism; and the processor receives at least one physiological signal output by the front-end circuit, determines whether there is a first physiological signal with a detection anomaly in the physiological signal, and when a detection anomaly occurs in the first physiological signal, determines an effective second physiological signal homologous to the first physiological signal, and obtains at least part of an analysis result corresponding to the first physiological signal based on the second physiological signal. When a detection anomaly occurs in the first physiological signal, the analysis result corresponding to the first physiological signal continues to be analyzed based on the second physiological signal, so that the analysis result corresponding to the first physiological signal is not interrupted, and continuous information is provided for medical care personnel.

### CROSS-REFERNECE OF RELATED APPLICATIONS

This application claims the benefits of priority to Chinese Application No. 202010990160.0, filed Sep. 18, 2020, the entire content of which is incorporated by reference in its entirety.

### TECHNICAL FIELD

The disclosure relates to a medical device, and in particular to a physiological parameter monitoring device and method.

### BACKGROUND

Medical monitoring refers to a process of detecting a physiological signal of an organism using a suitable sensor, and then performing an analysis based on the detected physiological signal to obtain an analysis result. After comparing the analysis result with a known set value, if the analysis result exceeds the known set value, an alarm may be further given. After continuously monitoring a physiological parameter of a patient, a medical monitor may further detect a trend and point out a critical status to provide a basis for a physician to deal with an emergency and carry out treatment. Therefore, monitoring physiological parameters for patients (especially critically ill patients) has been widely used in various hospitals. The physiological parameters monitored usually include body temperature, electrocardiogram (ECG), blood oxygen (SpO2), blood pressure, respiration, and electroencephalogram.

Currently, in monitoring of physiological parameters of a human body such as ECG monitoring, SpO2 monitoring, or blood pressure monitoring, generally each parameter is monitored individually, and respective analysis results are obtained based on the monitored physiological parameters, that is, an analysis result is obtained for each parameter based on the individual parameter. For example, an electrocardiogram-type analysis result is obtained based on an electrocardiogram signal, a blood-oxygen-type analysis result is obtained based on a blood oxygen signal, and a blood-pressure-type analysis result is obtained based on blood pressure. An analysis result may include a value or a waveform graph.

However, in a monitoring process, for some specific reasons, for example, because a sensor monitoring a physiological parameter falls off or is in poor contact, monitoring of the physiological parameter may be interrupted. During the interruption, information about one or more physiological indices obtained based on the physiological parameter may be lost, making it inconvenient for medical care personnel to determine a patient's condition. If it happens that the patient's condition changes during the interruption of monitoring and the patient needs to be administered, such a loss of the information caused by the interruption may pose a risk to the patient.

### SUMMARY

The disclosure mainly provides a physiological parameter monitoring device and method, which can reduce a risk brought to a patient due to interruption of physiological parameter monitoring.

According to a first aspect, an embodiment provides a physiological parameter monitoring device, including:

a front-end circuit configured to acquire a first physiological signal corresponding to a first physiological parameter of a patient from a signal output by at least one first sensor for sensing a physiological signal of the patient;

a processor configured to: receive at least one first physiological signal acquired by the front-end circuit, determine whether a detection anomaly occurs in the first physiological signal, and when it is determined that the detection anomaly occurs in the first physiological signal, determine whether there is an effective second physiological signal homologous to the first physiological signal, where the second physiological signal is output by at least one second sensor for sensing a physiological signal of the patient and corresponds to a second physiological parameter of the patient; and when it is determined that there is the second physiological signal, obtain at least part of information corresponding to the first physiological signal by using the second physiological signal, and analyze the first physiological parameter based on the at least part of information, to obtain an analysis result; and

an output component configured to receive the analysis result output by the processor, and output the analysis result in a perceivable manner.

According to a second aspect, an embodiment provides a physiological parameter monitoring device, including:

a front-end circuit configured to acquire a physiological signal from a signal output by at least one sensor for sensing a physiological signal of an organism, preprocess the acquired physiological signal and output a preprocessed physiological signal;

a processor configured to receive at least one physiological signal output by the front-end circuit, determine whether there is a first physiological signal with a detection anomaly in the physiological signal; when all physiological signals are normal, use the physiological signals to obtain their respective analysis results; when there is a first physiological signal with a detection anomaly, determine whether there is an effective second physiological signal homologous to the first physiological signal; and if there is an effective second physiological signal homologous to the first physiological signal, obtain at least part of information in an analysis result corresponding to the first physiological signal based on the second physiological signal, where homologous signals are defined to be signals derived from a vital activity of a same organ and detected and output by different sensors; and

an output component configured to receive the analysis result output by the processor, and output the analysis result in a perceivable manner.

According to a third aspect, an embodiment provides a physiological parameter monitoring method, including:

receiving at least one physiological signal output by at least one sensor for sensing a physiological signal of an organism;

determining whether there is a first physiological signal with a detection anomaly in the physiological signal;

when there is a first physiological signal with a detection anomaly, determining whether there is an effective second physiological signal homologous to the first physiological signal;

if there is an effective second physiological signal homologous to the first physiological signal, obtaining at least part of information in an analysis result corresponding to the first physiological signal based on the second physiological signal, where homologous signals are defined to be signals derived from a vital activity of a same organ and detected and output by different sensors; and

outputting an analysis result to an output component.

According to a fourth aspect, an embodiment provides a physiological parameter monitoring method, including:

receiving a first physiological signal output by a first sensor for sensing a physiological signal of an organism;

determining whether the first physiological signal is anomalous;

when the first physiological signal is anomalous, obtaining an effective second physiological signal homologous to the first physiological signal;

splicing the first physiological signal and the second physiological signal, to form a spliced signal; and

using the spliced signal to analyze a physiological parameter of the organism.

According to a fifth aspect, an embodiment provides a physiological parameter monitoring method, including:

receiving at least one physiological signal output by at least one sensor for sensing a physiological signal of an organism; determining whether there are homologous signals in the at least one physiological signal, where the homologous signals are defined to be signals derived from a vital activity of a same organ and detected and output by different sensors; and

if there are homologous signals in the at least one physiological signal, performing a fused analysis on at least two of the homologous signals, and obtaining at least part of information in an analysis result.

According to a sixth aspect, an embodiment provides a physiological parameter monitoring device, including:

a memory configured to store a program; and

a processor configured to execute the program to implement the method described above.

According to a seventh aspect, an embodiment provides a computer-readable storage medium including a program, the program being executable by a processor to implement the method described above.

In some embodiments, when a detection anomaly occurs in the first physiological signal, the effective second physiological signal homologous to the first physiological signal may be obtained, and the analysis result corresponding to the first physiological signal continues to be analyzed based on the second physiological signal, to obtain an analysis result, such that the analysis result corresponding to the first physiological signal is not interrupted, to provide continuous information for medical care personnel.

In some other embodiments, homologous signals are found from monitored physiological signals, a fused analysis is performed on at least two of the homologous signals, and at least part of information in an analysis result is obtained. When a physiological signal is anomalous, this solution can also make it possible that an analysis result corresponding to the anomalous physiological signal is not interrupted, to provide continuous information for the medical care personnel.

### BRIEF DESCRIPTION OF THE DRAWINGS

FIG. 1 is a system framework diagram of a physiological parameter monitoring device;

FIG. 2 shows waveform graphs of homologous signals on a same display interface;

FIG. 3 shows form changes of waveforms of homologous signals when a heart has a short heartbeat pause;

FIG. 4 shows form changes of waveforms of homologous signals when a heart failure and ventricular fibrillation occur;

FIG. 5 is a flowchart of replacing homologous signals with each other for analysis according to an embodiment;

FIG. 6a is a flowchart of analyzing a first physiological parameter by using homologous signals;

FIG. 6b is a schematic diagram of a relationship for replacing homologous signals with each other;

FIG. 7 is a flowchart of processing when a detection anomaly occurs in a monitoring process of an ECG signal;

FIG. 8 is a flowchart of processing when a detection anomaly occurs in a monitoring process of an SpO2 signal;

FIG. 9 is a flowchart of processing using both a correlation analysis and a rhythm analysis for homologous signals;

FIG. 10 is another flowchart of processing using both a correlation analysis and a rhythm analysis for homologous signals;

FIG. 11a is a diagram of splicing of homologous signals; and

FIG. 11b is a schematic diagram of a relationship for splicing homologous signals.

### DETAILED DESCRIPTIONS

The disclosure will be further described in detail below through specific implementations in conjunction with the accompanying drawings. Associated similar element reference numerals are used for similar elements in different implementations. In the following implementations, many details are described such that the disclosure may be better understood. However, it may be effortlessly appreciated by a person skilled in the art that some of the features may be omitted, or may be substituted by other elements, materials, and methods in different cases. In certain cases, some operations involved in the disclosure are not displayed or described in the specification, which is to prevent a core part of the disclosure from being obscured by too much description. Moreover, for a person skilled in the art, the detailed description of the involved operations is not necessary, and the involved operations can be thoroughly understood according to the description in the specification and general technical knowledge in the art.

In addition, the characteristics, operations, or features described in the specification may be combined in any appropriate manner to form various implementations. Meanwhile, the steps or actions in the method description may also be exchanged or adjusted in order in a way that is obvious to a person skilled in the art. Therefore, the various orders in the specification and the accompanying drawings are merely for the purpose of clear description of a certain embodiment and are not meant to be a necessary order unless it is otherwise stated that a certain order must be followed.

The serial numbers themselves for the components herein, for example, “first” and “second”, are merely used to distinguish described objects, and do not have any sequential or technical meaning. Moreover, as used in the disclosure, “connection” or “coupling”, unless otherwise stated, includes both direct and indirect connections (couplings).

The disclosure will be further described in detail below through specific implementations in conjunction with the accompanying drawings. Associated similar element reference numerals are used for similar elements in different implementations. In the following implementations, many details are described such that the disclosure may be better understood. However, it may be effortlessly appreciated by a person skilled in the art that some of the features may be omitted, or may be substituted by other elements, materials, and methods in different cases. In certain cases, some operations involved in the disclosure are not displayed or described in the specification, which is to prevent a core part of the disclosure from being obscured by too much description. Moreover, for a person skilled in the art, the detailed description of the involved operations is not necessary, and the involved operations can be thoroughly understood according to the description in the specification and general technical knowledge in the art.

In addition, the characteristics, operations, or features described in the specification may be combined in any appropriate manner to form various implementations. Meanwhile, the steps or actions in the method description may also be exchanged or adjusted in order in a way that is obvious to a person skilled in the art. Therefore, the various orders in the specification and the accompanying drawings are merely for the purpose of clear description of a certain embodiment and are not meant to be a necessary order unless it is otherwise stated that a certain order must be followed.

The serial numbers themselves for the components herein, for example, “first” and “second”, are merely used to distinguish described objects, and do not have any sequential or technical meaning. Moreover, as used in the disclosure, “connection” or “coupling”, unless otherwise stated, includes both direct and indirect connections (couplings).

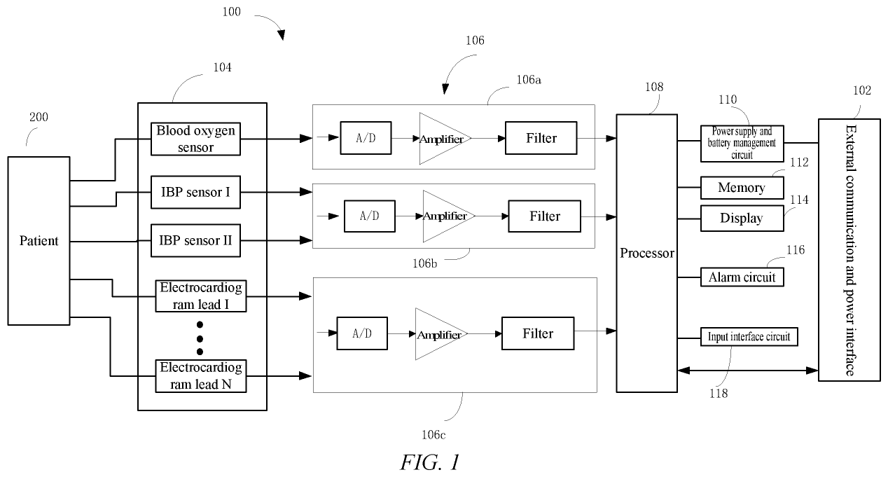

Referring to FIG. 1, FIG. 1 provides a system framework diagram of a physiological parameter monitoring device. The physiological parameter monitoring device is usually disposed beside a patient's bed, and is also referred to as a monitor. In a typical embodiment, the monitor is a multi-parameter monitor capable of monitoring a plurality of physiological parameters, and includes a housing, a main unit, and an output component. The output component may be a display for outputting an image, or a player for outputting a sound, or may be a light-emitting component for outputting light information.

A sensor interface area may be arranged on a housing panel. A plurality of sensor interfaces may be integrated in the sensor interface area and configured to be connected to various external physiological parameter sensor accessories 104. These physiological parameters include but are not limited to: parameters such as electrocardiogram, respiration, body temperature, blood oxygen, non-invasive blood pressure, and invasive blood pressure. When the monitor is an integrated device, an output component (such as a display) may also be provided on the housing panel; and when the monitor is a split-type device, the display and the main unit may be placed separately. An input interface circuit 118, an alarm circuit 116 (such as an LED alarm area), and the like may also be provided on the housing panel.

The main unit is located in the housing, and is configured to acquire and analyze a physiological parameter. In an embodiment, the main unit includes a front-end circuit 106 and a processor 108. The front-end circuit 106 is configured to acquire a physiological signal from a signal output by at least one sensor for sensing a physiological signal of an organism, preprocess the acquired physiological signal and then output a preprocessed physiological signal. In the embodiment shown in FIG. 1, the front-end circuit 106 includes a signal acquisition circuit, an amplifier, and a filter connected in sequence. The signal acquisition circuit may be, for example, an AID converter, which is configured to sample physiological signals detected by the physiological parameter sensor accessories 104, and convert an analog signal into a digital signal. The signal acquisition circuit may be selected from an electrocardiogram circuit, a respiration circuit, a body temperature circuit, a blood oxygen circuit, a non-invasive blood pressure circuit, an invasive blood pressure circuit, and the like. These signal acquisition circuits are electrically connected to respective sensor interfaces, so as to be electrically connected to sensor accessories 104 corresponding to different physiological parameters. Output digital signals are respectively amplified by the amplifier and denoised by the filter, and then output to the processor 108 for analysis and calculation. The sensor accessories 104 and the signal acquisition circuit 112 corresponding to various physiological parameters may use general-purpose circuits in the prior art.

In some embodiment, the front-end circuit 106 includes an amplifier, a filter, and a signal acquisition circuit connected in sequence. In other words, an input analog signal is first sampled, amplified, and filtered, and AID conversion is then performed to convert the analog signal into a digital signal.

The processor 108 is configured to receive at least one physiological signal output by each of front-end circuits 106a, 106b, and 106c, calculate and analyze the physiological signal, obtain a visual analysis result and output the analysis result to a display 114. After completing calculation of the physiological parameter, the processor 108 may further determine whether the physiological parameter is anomalous, and if so, it may give an alarm by means of the alarm circuit 116.

A memory 112 may store intermediate and final data of the monitor, and store program instructions or code to be executed by the processor 108 and the like. In this embodiment, the physiological signal output by the front-end circuit may also be cached in the memory 112.

If the monitor is available for non-invasive blood pressure measurement, it may further include a pump valve driving circuit configured to perform inflation or deflation operations under the control of the processor 108.

In some embodiments, the main unit may alternatively include a pluggable parameter processing module, the parameter processing module includes an interface that matches a corresponding physiological parameter sensor accessory and a front-end circuit configured to acquire and preprocess an input physiological parameter, the parameter processing module may further include a unit for calculating and analyzing the physiological parameter, and may output a calculation result and an analysis result to the processor, so that the processor may generate a visual analysis result and output the analysis result to the display. The parameter processing module has an external communication and power interface for communicating with the main unit and drawing power from the main unit, and may be connected to the main unit in a pluggable manner, to form a plug-in monitor main unit. The parameter processing module may alternatively be connected to the main unit by means of a cable and be used as an external accessory of the monitor.

The physiological parameter monitoring device may further include a power supply and battery management circuit 110, and the power supply and battery management circuit 110 draws power from an external or internal power supply by means of an external communication and power interface 102, and supplies power to the processor 108 after processing such as rectification and filtering. The external communication and power interface 102 may be one of or a combination of the Ethernet, a token ring, a token bus, and a local area network interface composed of a fiber distributed data interface (FDDI) for a backbone network of these three networks, or may be one of or a combination of wireless interfaces such as infrared, Bluetooth, Wi-Fi, and WMTS communication interfaces, or may be one of or a combination of wired data connection interfaces such as RS232 and USB interfaces. The external communication and power interface 102 may alternatively be one of a wireless data transmission interface and a wired data transmission interface or a combination thereof.

A physiological signal representing a vital sign of an organism is mainly derived from a physiological activity of an organ of the organism (such as a mechanical activity or bioelectrical activity), and the physiological activity is a basic characteristic of the organ of the organism. Physiological activities of the organ of the organism may be acquired by various sensors, to form various physiological parameters corresponding to the sensors, and the physiological parameters may be, for example, electrocardiogram parameters, electroencephalogram parameters, blood oxygen parameters, blood pressure parameters, respiration parameters, and muscle relaxation parameters. Generally, one type of sensor corresponds to one type of physiological parameter, and the physiological parameter includes a physiological signal acquired by the sensor and an analysis result obtained after the physiological signal is processed, analyzed, and calculated. That is, one type of sensor outputs one type of physiological parameter, the monitoring device analyzes the physiological parameter by means of an algorithm corresponding to the physiological parameter to generate various analysis results of the physiological parameter, and the analysis result may be a specific physiological index value or may be a waveform graph or a histogram. For example, an electrocardiogram parameter is obtained from a signal acquired through an electrocardiogram lead, an electroencephalogram parameter is obtained from a signal acquired through an electroencephalogram lead, a blood oxygen parameter is obtained from a signal acquired through a blood oxygen sensor, a blood pressure parameter is obtained from a signal acquired through a blood pressure meter, and the blood pressure parameter may alternatively be obtained in an invasive manner by inserting a probe into a blood vessel. Although physiological parameters obtained by different sensors are different, signals acquired by different sensors may have a same origin, that is, may be derived from physiological activities of a same organ, which are referred to as homologous signals. Homologous signals may be sensed by different types of sensors, or may be sensed by different detection channels of a same type of sensor. For example, an electrocardiogram signal acquired by the electrocardiogram lead reflects changes in a bioelectrical signal of a heart, a blood oxygen signal acquired by the blood oxygen sensor reflects a cardiac output of the heart, and a blood pressure signal in invasive blood pressure detection also reflects a pumping function of the heart. The origin of these signals is heart beat, that is, although the electrocardiogram signal (ECG), the blood oxygen signal (SpO2), and the invasive blood pressure signal (IBP) are obtained by different types of sensors, these signals are all related to mechanical motion of the heart. Therefore, the electrocardiogram signal, the blood oxygen signal, and the invasive blood pressure signal are homologous signals. The invasive blood pressure signal (IBP) usually has two detection channels. When invasive blood pressure signals are sensed by different detection channels of a same type of sensor, the invasive blood pressure signals of the two detection channels are homologous signals.

By analysis of the electrocardiogram signal, the blood oxygen signal, and the invasive blood pressure signal, a beating rhythm of the heart can be obtained. Because the homologous signals reflect physiological activities of a same organ of an organism, there should be a specific association among the homologous signals. The following uses the electrocardiogram signal (ECG), the blood oxygen signal (SpO2), and the invasive blood pressure signal (IBP) as an example to describe the association among the homologous signals.

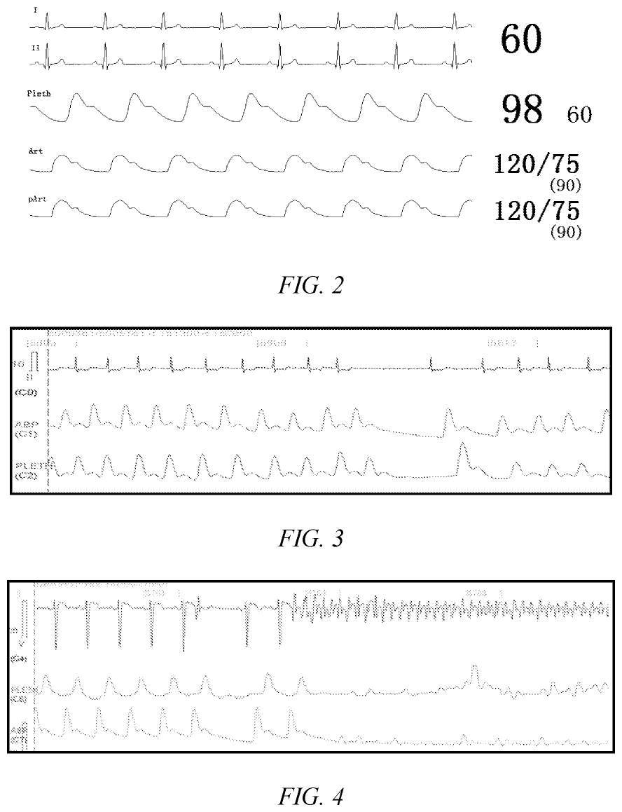

Referring to FIG. 2, when monitoring parameters include ECG, SpO2, and IBP, under normal conditions, a monitoring interface is as shown in FIG. 2. Waveforms corresponding to I and II in an upper area and a parameter value on the right are derived from ECG, where 60 on the right represents an HR value. A waveform corresponding to Pleth in a middle area and parameter values on the right are derived from SpO2, where 98 represents a blood oxygen value, and 60 represents a PR value. Waveforms and parameter values corresponding to Art and pArt on a bottom area are derived from IBP, where 120/75 represents systolic pressure and diastolic pressure, respectively, and 90 represents mean blood pressure.

In normal monitoring of parameters, because some of the parameters are homologous, for example, the three parameters, namely, ECG, SpO2, and IBP, are all derived from beating of a heart, theoretically, waveform changes of the three parameters are synchronous. For example, when a QRS complex occurs in the ECG parameter, a pulse wave peak also occurs in the SpO2 parameter and the IBP parameter basically at the same time, that is, the three parameters change synchronously. In addition, because the three parameters are all derived from the heart, when a cardiac function is impaired, it will be reflected in all the three parameters. By analysis of the three parameters, according to a parameter waveform and an analysis result, a correspondence between a form type and a result is established in a parameter model.

It has been found from experiments that when the heart has a short heartbeat pause, a continuous horizontal line of “heartbeat pause” appears on all the three parameter waveforms. As shown in FIG. 3, the waveforms from top to bottom are ECG, IBP, and SpO2.

When a heart failure and ventricular fibrillation occur, a fibrillation wave occurs in an ECG waveform, as shown in an upper waveform in FIG. 4, while there is almost no effective pulse wave in the SpO2 (middle waveform) and IBP (lower waveform) waveforms. A pumping function of the heart fails, and therefore there is weak or even no blood flow in the blood vessel.

It can be learned from the above that there is an association among an ECG signal, an IBP signal, and an SpO2 signal regarding rhythm information of the heart. Waveforms of the three signals have a same frequency but with a specific phase difference.

Based on the above knowledge, the inventor of the disclosure envisions using homology of signals for a fused analysis of the signals, e.g., replacing homologous signals with each other. For example, a first physiological signal and a second physiological signal are homologous signals. In a process of analyzing a first physiological parameter, for some physiological indices directly related to basic characteristics of an organ, under normal conditions, the first physiological signal is used to analyze the physiological indices. When a detection anomaly occurs in the first physiological signal, the inventor takes advantage of replaceability between the homologous signals and uses the second physiological signal to replace the first physiological signal to analyze these physiological indices directly related to the characteristics of the organ. For another example, the inventor envisions splicing homologous signals with each other. For example, the first physiological signal and the second physiological signal are homologous signals. When a detection anomaly occurs in the first physiological signal, the second physiological signal is spliced with the original first physiological signal to form a spliced signal, and a physiological parameter of the organism is analyzed based on the spliced signal. This makes it possible that, when the first physiological signal is anomalous (for example, a lead falls off or is in poor contact) but no detection anomaly occurs in the second physiological signal, the second physiological signal may continue to be used to analyze the first physiological parameter, and an analysis of the first physiological parameter may not necessarily be interrupted.

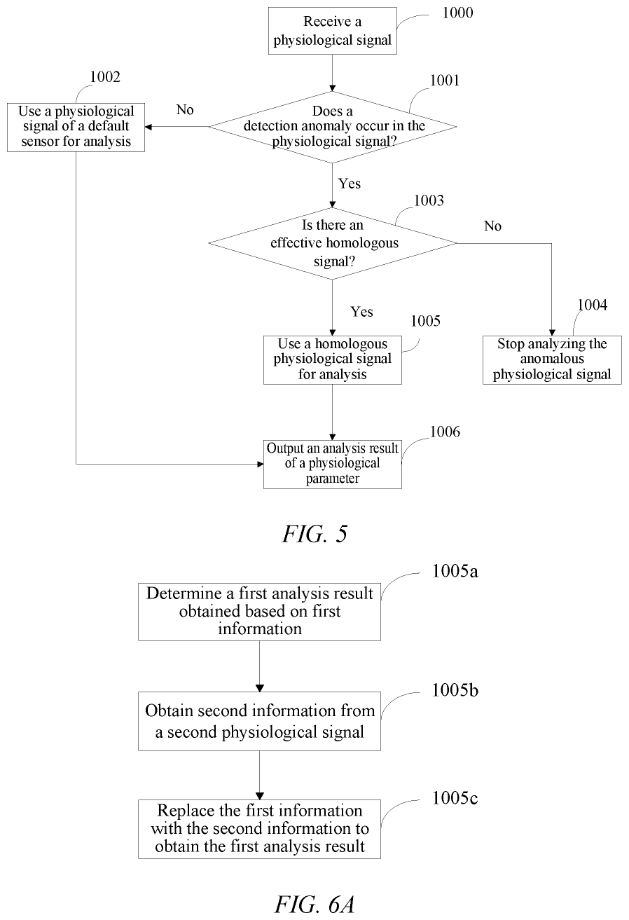

An embodiment shown in FIG. 5 illustrates how the homologous signals are replaced with each other.

In an embodiment, as shown in FIG. 5, a process of analyzing a physiological parameter by a processor includes the following steps.

Step 1000: Receive a physiological signal from the processor. The physiological signal may be directly output by a front-end circuit, or a cached physiological signal may be read from a memory. The processor performs corresponding algorithm processing on the physiological signal according to a type of the physiological signal, and obtains an analysis result of the physiological parameter. The processor may determine which physiological signals are homologous signals according to the type of the physiological signal. A determination method may be preset in a system. For example, an electrocardiogram signal, a blood oxygen signal, and an invasive blood pressure signal are predefined in the system to be homologous signals.

Step 1001: Determine whether a detection anomaly occurs in the physiological signal. In a monitoring process, the processor determines whether there is an anomalous physiological signal in a plurality of received physiological signals. Generally, the processor compares the physiological signal with a corresponding preset range, and when the physiological signal is beyond the preset range (for example, greater than an upper limit or less than a lower limit), the physiological signal is determined to be anomalous. Clinically, there are usually two causes for a physiological signal anomaly. One is that a patient's physical condition has changed, e.g., deterioration of the condition or interference (such as coughing or a limb being compressed) occurs, which causes corresponding changes to physiological signals of the body, and may cause a physiological signal detected by a sensor to exceed a normal range. Such a physiological signal anomaly is referred to as a physiological anomaly herein. After determining the physiological anomaly, the processor may generate a prompt signal or an alarm signal to indicate that the condition of the patient should be paid attention to. The other is that the patient's physical condition is not changed, but an anomaly occurs in detection, for example, a detection sensor falls off or is in poor contact, or the detection sensor is faulty, causing a signal to be interrupted or too large or too small, and the processor comes to a result that the physiological signal is anomalous. Such a physiological signal anomaly is referred to as a detection anomaly herein. This embodiment of the disclosure focuses on a detection anomaly. Therefore, in this embodiment, after determining that a physiological anomaly occurs, the processor further determines whether a detection anomaly occurs in a physiological signal. For example, when the processor determines that a physiological signal is beyond a preset range, the processor checks whether a homologous signal of the physiological signal is detected at the same time, and if so, it is determined, according to the homologous signal, whether a detection anomaly occurs in the physiological signal. For example, if the homologous signal is also beyond a preset range or has a consistent signal change at the same time, the physiological signal anomaly may be a physiological anomaly caused by a change of the patient's physical condition. If the homologous signal is not beyond the preset range or does not have a consistent signal change at the same time, the physiological signal anomaly may be a detection anomaly.

If all physiological signals are normal, step 1002 is performed: The processor performs normal calculation and analysis, performs corresponding algorithm processing on the physiological signal according to a type of the physiological signal, obtains an analysis result of the physiological parameter, and sends the analysis result to a display. In this step, the processor analyzes the physiological signal output by a default sensor. For example, when analyzing an electrocardiogram parameter, the processor uses a signal output by an electrocardiogram lead; when analyzing a blood oxygen parameter, the processor uses a signal output by a blood oxygen sensor; when analyzing a blood pressure parameter, the processor uses a signal output by an invasive blood pressure sensor or a blood pressure cuff; and when analyzing an electroencephalogram parameter, the processor uses a signal output by an electroencephalogram lead.

If a physiological anomaly occurs in a physiological signal, step 1002 is still performed, and prompt information or alarm information is output at the same time. If a detection anomaly occurs in a physiological signal, step 1003 is performed to determine whether there is an effective homologous signal of the physiological signal. In this step, the processor first confirms a homologous signal of the physiological signal with a detection anomaly, and then determines whether the homologous signal is effective. If a detection anomaly does not occur in the homologous signal, it is determined that the homologous signal is effective; and if a detection anomaly also occurs in the homologous signal, it is determined that the homologous signal is ineffective. There may be one or more homologous signals. When there are a plurality of homologous signals, it may be determined whether the homologous signals are effective one by one according to set priorities.

When all the homologous signals are ineffective, step 1004 is performed to stop analyzing the anomalous physiological signal, that is, the physiological parameter with a detection anomaly is no longer analyzed.

When there is an effective homologous signal, step 1005 is performed, and the homologous physiological signal is used to perform a fused analysis on the physiological parameter corresponding to the physiological signal with a detection anomaly. Because homologous signals reflect a vital activity of a same organ of an organism, such as a mechanical activity or bioelectrical activity of the organ of the organism, the homologous signals have similar trends, similar signal amplitude changes, or similar frequencies. Therefore, for physiological indices, waveform graphs, trend charts, etc., related to the vital activity of the organ of the organism in an analysis result, homologous signals can be used for a fused analysis.

For ease of explanation, it is assumed herein that the first physiological signal and the second physiological signal are homologous signals. The first physiological signal is detected by a first sensor and is used to analyze and calculate a first physiological parameter of the patient and to obtain an analysis result of the first physiological parameter. The second physiological signal is detected by a second sensor and is used to analyze and calculate a second physiological parameter of the patient and to obtain an analysis result of the second physiological parameter. When a detection anomaly occurs in the first physiological signal, if a detection anomaly does not occur in the second physiological signal, a fused analysis may be performed on the first physiological signal and the second physiological signal. There are two manners of the fused analysis: replacement and splicing.

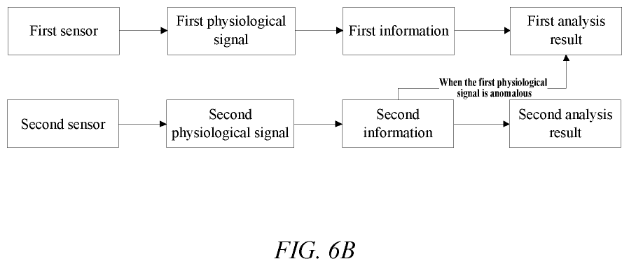

In a specific embodiment for replacement, the homologous second physiological signal is used to analyze and calculate the first physiological parameter of the patient and to obtain at least part of the analysis result of the first physiological parameter. A procedure thereof is shown in FIG. 6a and includes the following steps.

Step 1005a: Determine a first analysis result suitable for analysis by the homologous signal. Among analysis results of the first physiological parameter, some analysis results (such as specific physiological indices or waveform graphs) are related to an intensity or a period of a vital activity of the organ of the organism, and for these analysis results, homologous signals may be used for analysis. However, some analysis results are not related to the intensity or the period of the vital activity of the organ of the organism, but are related to other characteristics of the vital activity, which are not suitable for analysis directly by homologous signals. In this step, at least one first analysis result obtained based on first information is determined from the analysis results of the first physiological parameter according to a specific physiological index algorithm or analysis method, where the first information is an intermediate parameter that is obtained based on the first physiological signal and that can reflect a vital activity of the organ of the organism, for example, information that can reflect the intensity and/or the period of the vital activity of the organ of the organism, so as to obtain a specific physiological index that can be analyzed by using a homologous signal.

Step 1005b: Obtain second information from the second physiological signal, where the second information may be information that is obtained based on the second physiological signal and that can reflect the intensity of the vital activity of the organ of the organism, or may be information that is obtained based on the second physiological signal and that can reflect the period of the vital activity of the organ of the organism, such as rhythm information. The step of obtaining the rhythm information from the second physiological signal includes:

obtaining second signal peak information from the second physiological signal;

calculating first signal peak information of the first physiological signal based on the second signal peak information; and

obtaining rhythm information of the first physiological parameter based on the first signal peak information.

Step 1005c: Replace the first information with the second information to obtain the first analysis result. The first information and the second information are associated information of a same type, that is, both can reflect the intensity information of the vital activity of the organ of the organism, or both can reflect the period information of the vital activity of the organ of the organism. In a normal analysis process, the first analysis result is obtained based on the first information. When a detection anomaly occurs in the first physiological signal, the processor may replace the first information in the analysis process with the second information obtained from the homologous signal, to finally obtain the first analysis result. The process of replacing homologous signals with each other is shown in FIG. 6b.

In a specific embodiment, an analysis may be performed based on the second information and the first analysis result may be obtained. For those specific physiological indices that cannot be analyzed by using the second information, the processor may stop calculating or analyzing those specific physiological indices. In this case, the analysis result of the first physiological parameter includes only the first analysis result.

In another embodiment, an execution sequence of step 1005a and step 1005b can be changed.

Still referring to FIG. 5, after the analysis result of the first physiological parameter is obtained, step 1006 is performed to output the analysis result, for example, output the analysis result to a display or a printer for visual presentation. The analysis result may include specific physiological index values, or may be various graphs, such as waveform graphs, histograms, or pie charts.

The following uses the electrocardiogram signal (ECG), the blood oxygen signal (SpO2), and the invasive blood pressure signal (IBP) as an example to describe how homologous signals are replaced each other.

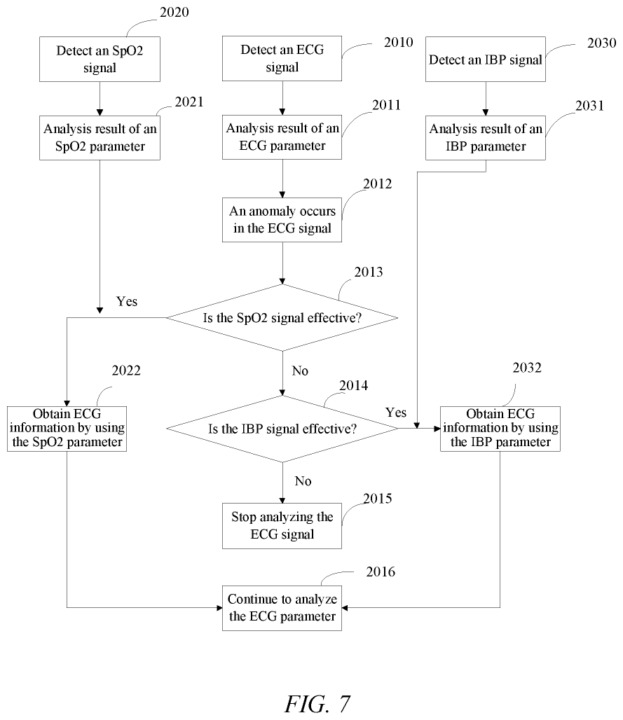

FIG. 7 shows a processing procedure when a detection anomaly occurs in a monitoring process of an ECG signal, including the following steps.

In step 2010, a processor receives an ECG signal detected by an electrocardiogram lead, and in step 2011, performs calculation and analysis based on the ECG signal to obtain an analysis result of an ECG parameter. The analysis result of the ECG parameter includes but is not limited to an ECG waveform, a heart rate (HR), an ST segment offset value and a QT segment related to the ECG waveform, and heart rate variability (HRV), arrhythmia (Arr), atrial fibrillation, and other analysis results based on an analysis of a heart rate.

In step 2020, the processor receives an SpO2 signal detected by a blood oxygen sensor, and in step 2021, performs calculation and analysis based on the SpO2 signal to obtain an analysis result of an SpO2 parameter. The analysis result of the SpO2 parameter includes but is not limited to a blood oxygen value (SpO2 value), a pulse rate (PR), and a perfusion index (PI).

In step 2030, the processor receives an IBP signal detected by an invasive blood pressure probe, and in step 2031, performs calculation and analysis based on the IBP signal to obtain an analysis result of an IBP parameter. The analysis result of the IBP parameter includes but is not limited to blood pressure (including systolic pressure, diastolic pressure, and mean blood pressure) and a pulse rate (PR).

In step 2012, the processor determines whether the ECG signal is anomalous. For example, when the ECG lead falls off or is in poor contact, or a sensor is faulty, monitoring of the ECG signal may be interrupted. At the same time, the processor detects that the SpO2 signal and/or the IBP signal are/is not interrupted. Therefore, it may be determined that a detection anomaly occurs in the ECG signal. When a detection anomaly occurs in the ECG signal, the processor performs step 2013.

In step 2013, the processor determines whether the SpO2 signal is effective, and when a detection anomaly does not occur in the SpO2 signal, it is determined that the SpO2 signal is currently effective, and step 2022 is performed. When a detection anomaly occurs in the SpO2 signal, it is determined that the SpO2 signal is currently ineffective, and step 2014 is performed.

In step 2022, the processor obtains ECG information by using the SpO2 parameter. Analysis results such as heart rate variability (HRV), arrhythmia (Arr), and atrial fibrillation in step 2011 are all based on a heart rate. The heart rate is related to a beating rhythm of the heart, and refers to the number of heartbeats per minute. Therefore, a pulse rate may be extracted from the SpO2 parameter, and the pulse rate refers to the number of detected pulses per minute, and is also related to the beating rhythm of the heart. Therefore, the pulse rate may be used to replace the heart rate in the ECG parameter. In step 2016, the ECG parameter continues to be analyzed to obtain analysis results such as heart rate variability (HRV), arrhythmia (Arr), and atrial fibrillation. In step 2014, the processor determines whether the IBP signal is effective, and when a detection anomaly does not occur in the IBP signal at this time, it is determined that the IBP signal is currently effective, and step 2032 is performed. When a detection anomaly occurs in the IBP signal at this time, it is determined that the IBP signal is currently ineffective, and step 2015 is performed to stop analyzing the ECG parameter.

In step 2032, the processor obtains ECG information by using the IBP parameter. The analysis result of the IBP parameter also includes the pulse rate (PR), and therefore, the pulse rate may be used to replace the heart rate in the ECG parameter. In step 2016, the ECG parameter continues to be analyzed to obtain the analysis result.

In step 2016, under normal conditions, the heart rate is obtained based on an ECG waveform, and then the heart rate variability (HRV) is obtained based on the heart rate, or the heart rate is analyzed to obtain the arrhythmia (Arr). When a detection anomaly occurs in the ECG signal, a pulse rate obtained from an SpO2 waveform or an IBP waveform may be used to replace the heart rate, and the analysis results of the heart rate variability and arrhythmia may be further obtained, so that values of the heart rate (HR) and the heart rate variability (HRV) and an analysis result of the arrhythmia (Arr) may continue to be displayed on a display interface, and display of this physiological index is not interrupted.

In the embodiment shown in FIG. 7, it is shown that whether the SpO2 signal is effective is first determined and whether the IBP signal is effective is then determined. Those skilled in the art may understand that in other embodiments, it is also possible to first determine whether the IBP signal is effective and then determine whether the SpO2 signal is effective.

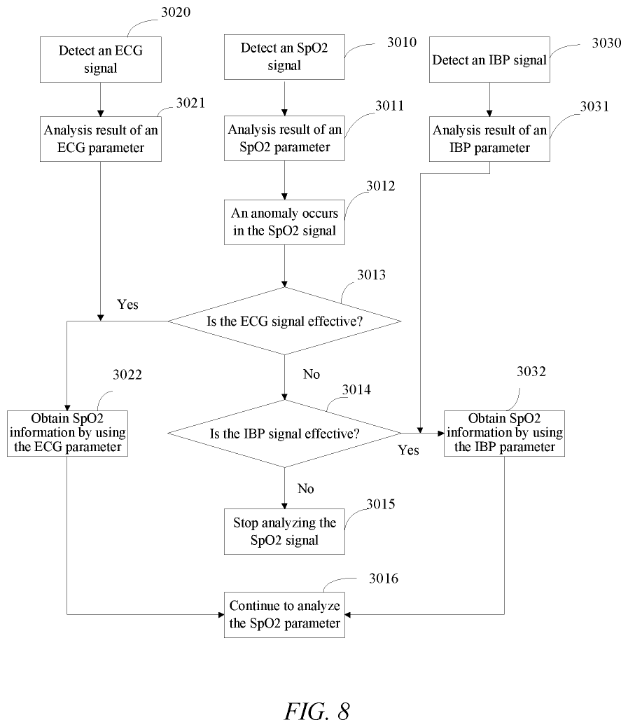

FIG. 8 shows a processing procedure when a detection anomaly occurs in a monitoring process of an SpO2 signal, including the following steps.

In step 3010, a processor receives an SpO2 signal detected by a blood oxygen sensor, and in step 3011, performs calculation and analysis based on the SpO2 signal to obtain an analysis result of an SpO2 parameter.

In step 3020, the processor receives an ECG signal detected by an electrocardiogram lead, and in step 3021, performs calculation and analysis based on the ECG signal to obtain an analysis result of an ECG parameter.

In step 3030, the processor receives an IBP signal detected by an invasive blood pressure probe, and in step 3031, performs calculation and analysis based on the IBP signal to obtain an analysis result of an IBP parameter.

In step 3012, the processor determines whether the SpO2 signal is anomalous. When a detection anomaly occurs in the SpO2 signal, the processor performs step 3013.

In step 3013, the processor determines whether the ECG signal is effective, and when a detection anomaly does not occur in the ECG signal, it is determined that the ECG signal is currently effective, and step 3022 is performed. When a detection anomaly occurs in the ECG signal, it is determined that the ECG signal is currently ineffective, and step 3014 is performed to continue to determine whether the IBP signal is effective. When a detection anomaly does not occur in the IBP signal, it is determined that the IBP signal is currently effective, and step 3032 is performed. When a detection anomaly occurs in the IBP signal, it is determined that the IBP signal is currently ineffective, and step 3015 is performed to stop analyzing the SpO2 parameter.

In step 3022, the processor obtains SpO2 information by using the ECG parameter. In step 3032, the processor obtains SpO2 information by using the IBP parameter. When monitoring interruption occurs in the SpO2 parameter, the ECG parameter or IBP parameter needs to be effective to implement a continuous SpO2 analysis. For example, during interruption of the SpO2 parameter, the ECG parameter or the IBP parameter is still being monitored, and the SpO2 parameter is automatically switched to the ECG parameter or the IBP parameter for rhythm analysis, to continue to analyze a rhythm of SpO2, for example, calculating a PR value. However, if the ECG parameter and the IBP parameter are also interrupted during the interruption of the SpO2 parameter, an analysis of the rhythm of the SpO2 is also interrupted.

In step 3016, under normal conditions, a pulse rate is obtained based on an SpO2 waveform. When a detection anomaly occurs in the SpO2 signal, a heart rate obtained based on an ECG waveform or a pulse rate obtained based on an IBP waveform may be used to replace the pulse rate in the SpO2 parameter, and a pulse rate value may continue to be displayed in a blood oxygen display area on the display interface, so that display of this physiological index is not interrupted.

In the embodiment shown in FIG. 8, it is shown that whether the ECG signal is effective is first determined and whether the IBP signal is effective is then determined. Those skilled in the art may understand that in other embodiments, it is also possible to first determine whether the IBP signal is effective and then determine whether the ECG signal is effective.

The IBP signal may be a single-channel signal or a dual-channel signal. When the IBP signal is a dual-channel signal, a channel having better signal quality is selected for the above analysis according to signal quality of two IBP channels. Details include:

determining whether the two channels of invasive blood pressure signals are normal, and if only one channel of invasive blood pressure signal is normal, using the normal invasive blood pressure signal as the second physiological signal, or if both the channels of invasive blood pressure signals are normal, using one channel of invasive blood pressure signal having better signal quality as the second physiological signal.

For the IBP signal, if the IBP detection is single-channel, when monitoring interruption occurs in the IBP signal due to a detection anomaly, information reflecting a heart rhythm in the ECG parameter or the SpO2 parameter may be used to continue to analyze the IBP parameter to obtain an analysis result of the IBP parameter. Details include the following steps:

determining whether an effective electrocardiogram signal and/or blood oxygen signal is detected; and if only one of the electrocardiogram signal and the blood oxygen signal is effective, using the effective signal as the second physiological signal; or if both the electrocardiogram signal and the blood oxygen signal are effective, selecting one of the electrocardiogram signal and the blood oxygen signal as the second physiological signal; or if it is detected that both the electrocardiogram signal and the blood oxygen signal are ineffective, determining that there is no second physiological signal.

If the IBP detection is dual-channel, different processing may be performed depending on whether a detection anomaly occurs in the two channels of invasive blood pressure signals, which may be divided into two cases.

In a first case, when a detection anomaly occurs in both the channels: in an embodiment, if only one of the electrocardiogram signal and the blood oxygen signal is effective, the effective signal is used as the second physiological signal to continue to perform a rhythm analysis; if both the electrocardiogram signal and the blood oxygen signal are effective, information reflecting a heart rhythm in the ECG parameter or the SpO2 parameter may be used to continue to analyze the IBP parameter to obtain an analysis result of the IBP parameter; and if it is detected that both the electrocardiogram signal and the blood oxygen signal are ineffective, it is determined that there is no second physiological signal.

In a second case, if a detection anomaly occurs in only one channel of invasive blood pressure signal, the channel of invasive blood pressure signal with a detection anomaly is used as the first physiological signal, and the other channel of invasive blood pressure signal is used as the second physiological signal to continue to perform a rhythm analysis. Certainly, an effective electrocardiogram signal or blood oxygen signal may also be used as the second physiological signal, and a rhythm analysis may continue to be performed.

According to the foregoing description, when a detection anomaly occurs in the first physiological signal, an effective homologous signal thereof may be used to replace the first physiological signal for a rhythm analysis, or an analysis result based on rhythm information may continue to be analyzed.

In another embodiment, when a detection anomaly occurs in the first physiological signal, in one aspect, rhythm information in the first physiological parameter may be analyzed by using rhythm information reflecting a vital activity of a same organ in the homologous signal, and in another aspect, a parameter value other than rhythm information may be predicted by means of a correlation analysis of the first physiological parameter and its homologous signal. Main steps thereof include:

determining a parameter model according to a physiological parameter form type corresponding to the second physiological signal, where the parameter model includes a correspondence between a physiological parameter form type and a predicted physiological parameter; and

determining a physiological parameter corresponding to the first physiological signal according to the parameter model.

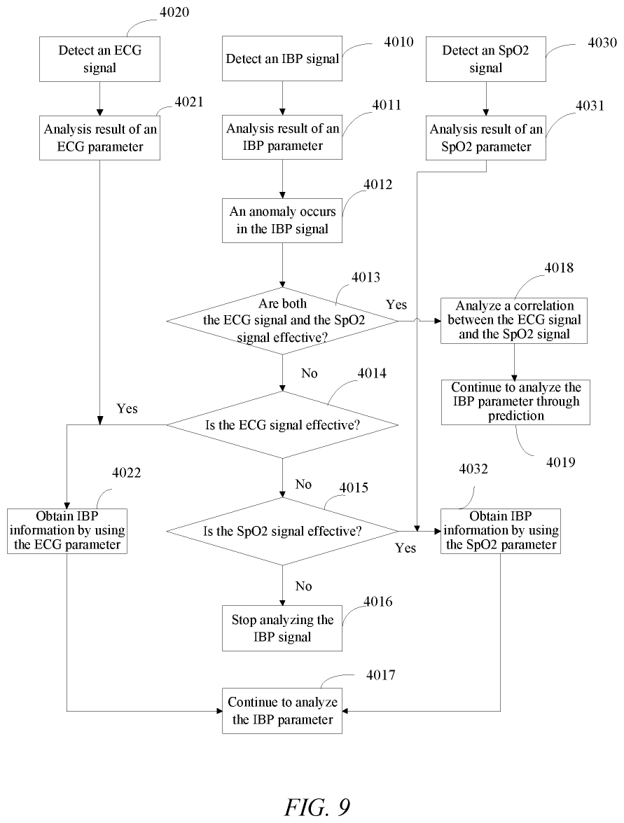

For example, when the two channels of IBP parameters are interrupted, if both the ECG parameter and the SpO2 parameter are effective, in one aspect, blood pressure values of the two IBP channels may be inferred according to a correlation between an ECG waveform and an SpO2 waveform, and in another aspect, the ECG parameter or SpO2 parameter may be used to continue to analyze a rhythm of IBP (for example, calculating a PR value), so as to implement an uninterrupted analysis of the IBP parameter. If one of the ECG parameter and the SpO2 parameter is effective, the ECG parameter or the SpO2 parameter continues to be used to perform a rhythm analysis on an IBP parameter. If both ECG parameter and the SpO2 parameter are interrupted, an analysis of an IBP parameter is also interrupted. A flowchart of an embodiment is shown in FIG. 9 and the embodiment includes the following steps.

In step 4010, a processor receives an IBP signal detected by an invasive blood pressure probe, and in step 4011, performs calculation and analysis based on the IBP signal to obtain an analysis result of an IBP parameter.

In step 4020, the processor receives an ECG signal detected by an electrocardiogram lead, and in step 4021, performs calculation and analysis based on the ECG signal to obtain an analysis result of an ECG parameter.

In step 4030, the processor receives an SpO2 signal detected by a blood oxygen sensor, and in step 4031, performs calculation and analysis based on the SpO2 signal to obtain an analysis result of an SpO2 parameter.

In step 4012, the processor determines whether the IBP signal is anomalous. When a detection anomaly occurs in the IBP signal, the processor performs step 4013.

In step 4013, the processor determines whether the ECG signal and the SpO2 signal are both effective; and if the ECG signal and the SpO2 signal are both effective, performs step 4018; or if not both of the ECG signal and the SpO2 signal are effective, performs step 4014.

In step 4018, a correlation between the ECG signal and the SpO2 signal is analyzed, and at the same time, one of the ECG signal and the SpO2 signal may be selected for a rhythm information analysis.

In step 4019, the IBP parameter continues to be analyzed through prediction according to the correlation obtained in step 4018. For example, blood pressure values of the two IBP channels may be inferred according to a conduction time difference (deltaT) between the ECG waveform and the SpO2 waveform, an IBP monitoring position, a change trend of deltaT and a change trend of the blood pressure values of the two IBP channels, etc.

In step 4014, the processor determines whether the ECG signal is effective; and if the ECG signal is effective, performs step 4022; or if the ECG signal is not effective, performs step 4015. Those skilled in the art should understand that in this step, it is also possible to first determine whether the SpO2 signal is currently effective.

In step 4022, the processor obtains IBP information by using the ECG parameter. The pulse rate in the IBP parameter is related to a beating rhythm of the heart, and the heart rate in the ECG parameter is also related to the beating rhythm of the heart. Therefore, the heart rate may be obtained from the ECG parameter to replace the pulse rate in the IBP parameter.

In step 4015, the processor continues to determine whether the SpO2 signal is effective. When a detection anomaly does not occur in the SpO2 signal, it is determined that the SpO2 signal is currently effective, and step 4032 is performed. When a detection anomaly occurs in the SpO2 signal, it is determined that the IBP signal is currently ineffective, and step 4016 is performed to stop analyzing the IBP parameter.

In step 4022, the processor obtains IBP information by using the SpO2 parameter. For example, the pulse rate in the SpO2 parameter is obtained to replace the pulse rate in the IBP parameter.

In step 4017, under normal conditions, a pulse rate is obtained based on an IBP waveform. When a detection anomaly occurs in the IBP signal, a heart rate obtained based on an ECG waveform or a pulse rate obtained based on an SpO2 waveform may be used to replace the pulse rate in the IBP parameter, and a pulse rate value may continue to be displayed in a blood oxygen display area on the display interface, so that display of this physiological index is not interrupted.

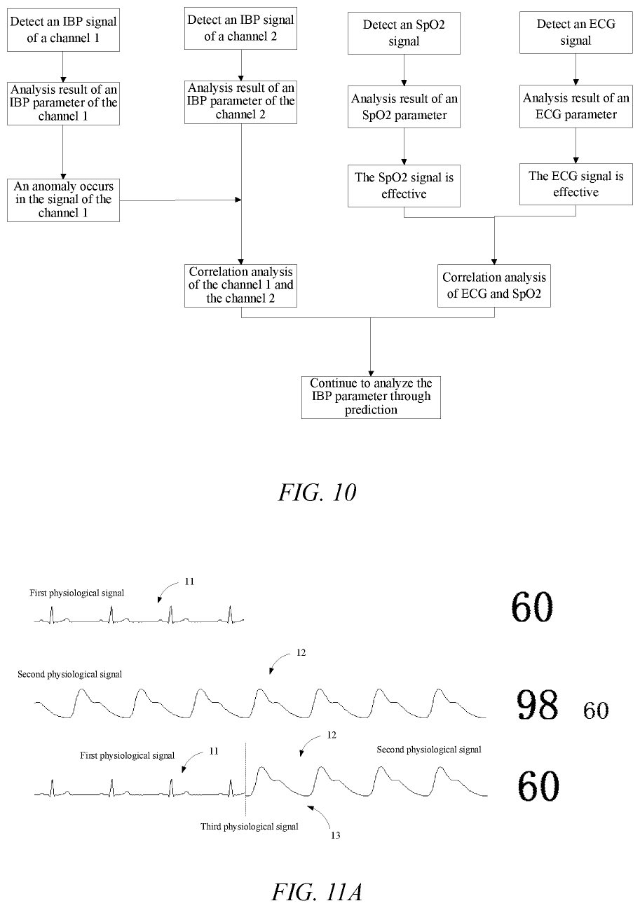

For another example, if a detection anomaly occurs in one of the two channels, that is, when monitoring detection occurs in one channel of IBP parameters, a correlation between blood pressure in the two channels and a correlation between an ECG parameter and an SpO2 parameter are first analyzed. Factors that affect blood pressure in the two channels include: monitoring positions of the two IBP channels, a blood pressure change trend when monitoring with both the two IBP channels, etc. Factors that affect the correlation between an ECG parameter and an SpO2 parameter include a conduction time difference deltaT, a change trend of deltaT, a change trend of blood pressure of an interrupted IBP channel, etc., and then a blood pressure value of the interrupted channel is inferred according to the correlation. In addition, a rhythm analysis (for example, calculating a PR value) continues to be analyzed according to an uninterrupted IBP channel. If the ECG parameter or the SpO2 parameter is also interrupted, only a correlation between the blood pressure of the two IBP channels is used to infer a blood pressure value of the interrupted channel to achieve continuous monitoring of the blood pressure and rhythm of the two channels. A flowchart is shown in FIG. 10.

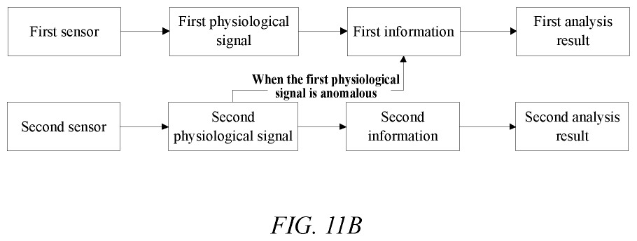

The other manner of a fused analysis is splicing. As shown in FIG. 11a, when a detection anomaly occurs on a first physiological signal 11, a homologous and effective second physiological signal 12 is directly spliced with the first physiological signal 11 to form a spliced signal 13 that can be displayed continuously, and the spliced signal 13 is subsequently used to analyze a physiological parameter of an organism. In other words, the spliced signal 13 includes two parts, one part is the first physiological signal 11 before the detection anomaly occurs, and the other part is the second physiological signal 12 after the detection anomaly occurs. For example, the first physiological signal 11 is an ECG signal, and the second physiological signal 12 is a blood oxygen signal.

A relationship for a fused analysis of the first physiological signal and the second physiological signal is shown in FIG. 11b. During calculation of information reflecting an intensity and/or a period of a vital activity of an organ of an organism (such as rhythm information of the heart) corresponding to the first physiological signal, before the detection anomaly occurs, the information is calculated by using the first physiological signal 11, and after the detection anomaly occurs, the information is calculated by using the second physiological signal 12 in the spliced signal 13.

Because phases of the first physiological signal 11 and the second physiological signal 12 are different, in order to eliminate an error, the information reflecting the intensity and/or the period of the vital activity of the organ of the organism may be calculated after a few periods of interruption, that is, several periods of newly spliced signals are discarded.

In the above embodiment, it is first determined whether a detection anomaly occurs in a physiological signal, and then it is determined whether there is a homologous signal of the signal with a detection anomaly. In other embodiments, the following execution steps may alternatively be used:

receiving at least one physiological signal output by at least one sensor for sensing a physiological signal of an organism;

determining whether there are homologous signals in the at least one physiological signal, where the homologous signals are derived from a vital activity of a same organ and is detected and output by different sensors; and

if there are homologous signals, performing a fused analysis on at least two of the homologous signals and obtaining at least part of an analysis result. A process of the fused analysis includes the following steps:

performing a signal quality analysis on the at least two of the homologous signals, and determining whether there is a first physiological signal with a detection anomaly; when there is a first physiological signal with a detection anomaly, determining whether a homologous signal of the first physiological signal is effective; and obtaining at least part of an analysis result corresponding to the first physiological signal based on the effective homologous signal.

Those skilled in the art may understand that all or some of the functions of the various methods in the above implementations may be implemented by means of hardware or by means of a computer program. When all or some of the functions in the above implementations are implemented by means of a computer program, the program may be stored in a computer-readable storage medium, and the storage medium may include: a read-only memory, a random access memory, a magnetic disk, an optical disk, a hard disk, and the like, and the program is executed by a computer to implement the above functions. For example, the program is stored in a memory of an apparatus, and when the program in the memory is executed by means of a processor, all or some of the above functions can be implemented. In addition, when all or some of the functions in the above implementations are implemented by means of a computer program, the program may also be stored in a storage medium such as a server, another computer, a magnetic disk, an optical disk, a flash disk, or a mobile hard disk, may be saved in a memory of a local apparatus through downloading or copying, or version updating may be performed on a system of the local apparatus. When the program in the memory is executed by means of a processor, all or some of the functions in the above implementations can be implemented.

The disclosure has been described by using specific examples above, which are merely for the purpose of facilitating understanding of the disclosure and are not intended to limit the disclosure. For a person of ordinary skill in the art, changes may be made to the above specific implementations according to the idea of the disclosure.

1-41. (canceled)

42. A physiological parameter monitoring device, comprising:
a front-end circuit configured to acquire a first physiological signal with a detection anomaly corresponding to a first physiological parameter of a patient from a signal output by a first sensor and a second physiological signal without a detection anomaly corresponding to a second physiological parameter of the patient output by a second sensor, wherein the first physiological signal and the second physiological signal are homologous signals, the homologous signals being signals derived from a vital activity of a same organ;
a processor configured to receive the first physiological signal and the second physiological signal, and obtain at least part of a first analysis result of the first physiological parameter at least partially based on the second physiological signal; and
an output component configured to receive the analysis result output by the processor, and output the analysis result in a perceivable manner.

43. The physiological parameter monitoring device of claim 42, wherein to obtain the at least part of the first analysis result, the processor is further configured to:
obtain, at least partially from the second physiological signal, information related to the first physiological parameter and reflecting an intensity and/or a period of a vital activity of the organ; and
obtain the first analysis result based on the information related to the first physiological parameter;
or, wherein to obtain the at least part of the first analysis result, the processor is further configured to:
select a first part of the first analysis result obtained based on first information, wherein the first information is obtained based on the first physiological signal and reflects an intensity or a period of a vital activity of the organ;
obtain second information at least partially from the second physiological signal, wherein the second information reflects the intensity or period of the vital activity of the organ; and
replace the first information with the second information to update the first analysis result.

44. The physiological parameter monitoring device of claim 43, wherein the first physiological signal and the second physiological signal are any two of an electrocardiogram signal, a blood oxygen signal, and an invasive blood pressure signal.

45. The physiological parameter monitoring device of claim 43, wherein the information related to the first physiological parameter, the first information and the second information are rhythm information of a heart.

46. The physiological parameter monitoring device of claim 45, wherein to obtain the at least part of the first analysis result, the processor is further configured to:
obtaining second signal peak information from the second physiological signal;
calculating first signal peak information of the first physiological signal based on the second signal peak information; and
obtaining rhythm information of the first physiological parameter based on the first signal peak information.

47. The physiological parameter monitoring device of claim 44, wherein the first physiological signal is an electrocardiogram signal and the second physiological signal is a blood oxygen signal, or the second physiological signal is an invasive blood pressure signal if no blood oxygen signal without a detection anomaly is detected.

48. The physiological parameter monitoring device of claim 44, wherein the first physiological signal is a blood oxygen signal and the second physiological signal is an electrocardiogram signal, or the second physiological signal is an invasive blood pressure signal if no electrocardiogram signal without a detection anomaly is detected.

49. The physiological parameter monitoring device of claim 44, wherein the first physiological signal is a blood oxygen signal or an electrocardiogram signal; the invasive blood pressure signal comprises a first channel signal and a second channel signal; and the second physiological signal is the second channel signal if the first channel signal is anomalous or has a weaker signal quality than the second channel signal.

50. The physiological parameter monitoring device of claim 44, wherein the first physiological signal is an invasive blood pressure signal consisting of a first channel signal with the detection anomaly and the second physiological signal is an electrocardiogram signal or blood oxygen signal, or
wherein the first physiological signal is an invasive blood pressure signal consisting of a first channel signal with the detection anomaly and a second channel signal without the detection anomaly and the second physiological signal is the second channel signal.

51. The physiological parameter monitoring device of claim 42, wherein to obtain at least part of a first analysis result of the first physiological parameter based on the second physiological signal, the processor is further configured to:
determine a parameter model comprising a correspondence between the first physiological parameter and a physiological parameter form type of the second physiological signal; and
determine the first physiological parameter according to the parameter model.

52. The physiological parameter monitoring device of claim 42, wherein the processor is further configured to check whether a homologous signal of the first physiological signal changes consistently when the first physiological signal is interrupted or changes.

53. A physiological parameter monitoring method, comprising:
receiving a first physiological signal with a detection anomaly corresponding to a first physiological parameter of a patient from a signal output by a first sensor and a second physiological signal without a detection anomaly corresponding to a second physiological parameter of the patient output by a second sensor, wherein the first physiological signal and the second physiological signal are homologous signals, the homologous signals being signals derived from a vital activity of a same organ;
obtaining at least part of a first analysis result of the first physiological parameter at least partially based on the second physiological signal; and
outputting the first analysis result to an output component.

54. The physiological parameter monitoring method of claim 53, wherein obtaining the at least part of the first analysis result comprises:
obtaining, at least partially from the second physiological signal, information related to the first physiological parameter and reflecting an intensity and/or a period of a vital activity of the organ; and
obtaining the first analysis result based on the information related to the first physiological parameter;
or, wherein obtaining the at least part of the first analysis result comprises:
selecting a first part of the first analysis result obtained based on first information, wherein the first information is obtained based on the first physiological signal and reflects an intensity or a period of a vital activity of the organ;
obtaining second information at least partially from the second physiological signal, wherein the second information reflects the intensity or period of the vital activity of the organ; and
replacing the first information with the second information to update the first analysis result.

55. The physiological parameter monitoring method of claim 54, wherein the first physiological signal and the second physiological signal are any two of an electrocardiogram signal, a blood oxygen signal, and an invasive blood pressure signal.

56. The physiological parameter monitoring method of claim 55, wherein the first physiological signal is an electrocardiogram signal the second physiological signal is a blood oxygen signal, or the second physiological signal is an invasive blood pressure signal if no blood oxygen signal without a detection anomaly is detected.

57. The physiological parameter monitoring method of claim 55, wherein the first physiological signal is a blood oxygen signal the second physiological signal is an electrocardiogram signal, or the second physiological signal is an invasive blood pressure signal if no electrocardiogram signal without a detection anomaly is detected.

58. The physiological parameter monitoring method of claim 55, wherein the first physiological signal is an invasive blood pressure signal consisting of a first channel signal with the detection anomaly and the second physiological signal is an electrocardiogram signal or blood oxygen signal, or
wherein the first physiological signal is an invasive blood pressure signal consisting of a first channel signal with the detection anomaly and a second channel signal without the detection anomaly and the second physiological signal is the second channel signal.

59. The physiological parameter monitoring method of claim 53, wherein obtaining at least part of a first analysis result of the first physiological parameter based on the second physiological signal comprises:
determining a parameter model comprising a correspondence between the first physiological parameter and a physiological parameter form type of the second physiological signal; and
determining the first physiological parameter according to the parameter model.

60. The physiological parameter monitoring method of claim 53, further comprising:
checking whether a homologous signal of the first physiological signal changes consistently when the first physiological signal is interrupted or changes.

61. The physiological parameter monitoring method of claim 53, wherein obtaining at least part of a first analysis result of the first physiological parameter based on the second physiological signal comprises:
splicing or fusing the first physiological signal and the second physiological signal, to form a spliced or fused signal; and
using the spliced or fused signal to obtain at least part of the first analysis result of the first physiological parameter.
